(19) United States
(12) Patent Application Publication
(10) Pub. No.: US 2004/0260374 A1
(43) Pub. Date: Dec. 23, 2004

(54) Implantable lead with fixation mechanism in the pulmonary artery

(75) Inventors: Yongxing Zhang, Little Canada, MN (US); Christopher P. Knapp, Ham Lake, MN (US)

Correspondence Address: Schwegman, Lundberg, Woessner & Kluth, P.A., P.O. Box 2938, Minneapolis, MN, 55402, US

(73) Assignee: Cardiac Pacemakers, Inc.

(21) Appl. No.: 10/895,747

(22) Filed: Jul. 21, 2004

Related U.S. Application Data
(63) Continuation-in-part of application No. 10/325,658, filed on Dec. 19, 2002.

(51) Int. Cl. A61N 1/05

(52) U.S. Cl. 607/122

(57) ABSTRACT

A lead body extends from a proximal end to a distal end and includes an intermediate portion and an electrode disposed along the intermediate portion. The distal end of the lead includes a pre-formed, biased shape adapted to passively fixate the distal end of the lead within a pulmonary artery with the electrode positioned against the ventricular septum or ventricular outflow tract. The lead body can include a curved portion and a second electrode disposed along the curved portion, wherein the second electrode is positioned a distance from the first electrode such that the second electrode is within the right atrium when the first electrode is positioned against the ventricular septum or ventricular outflow tract.

### RELATED APPLICATION

This application is a continuation-in-part and claims priority of invention under 35 U.S.C. §120 from U.S. application Ser. No. 10/325,658, filed Dec. 19, 2002, which is incorporated herein by reference.

### FIELD

This invention relates to the field of medical leads, and more specifically to an implantable lead.

### BACKGROUND

Leads implanted in or about the heart have been used to reverse certain life threatening arrhythmia, or to stimulate contraction of the heart. Electrical energy is applied to the heart via an electrode to return the heart to normal rhythm. Leads are usually positioned in the ventricle or in the atrium through a subclavian vein, and the lead terminal pins are attached to a pacemaker which is implanted subcutaneously.

For example, one approach is to place the electrode against the ventricular septum above the apex. However, current leads require a lead placed with the electrode against the septum above the apex to be actively fixated. This may possibly result in trauma to the heart from cyclical heart motion, and lead to micro-dislodgement of the electrode, and relatively higher defibrillating and pacing thresholds. Moreover, other factors which can be improved include better electrode contact, and easier implanting and explanting of the leads. Also, there is a need for leads designed for better delivery of therapy for cardiac heart failure (CHF).

### SUMMARY

One aspect includes a lead body extending from a proximal end to a distal end and having an intermediate portion and an electrode disposed along the intermediate portion of the lead. The distal end of the lead includes a pre-formed, biased shape adapted to passively fixate the distal end of the lead within a pulmonary artery with the electrode positioned against the ventricular septum or ventricular outflow tract. The lead body also includes a curved portion and a second electrode disposed along the curved portion, wherein the second electrode is positioned a distance from the first electrode such that the second electrode is within the right atrium when the first electrode is positioned against the ventricular septum or ventricular outflow tract.

A further aspect includes a lead body extending from a proximal end to a distal end and having an intermediate portion and at least two electrodes disposed along the intermediate portion of the lead. The distal end of the lead body is adapted to be fixated within a pulmonary artery, such that the at least two electrodes are located proximate a ventricular septum or ventricular outflow tract.

### BRIEF DESCRIPTION OF THE DRAWINGS

FIG. 1 shows a view of a lead, according to one embodiment, implanted within a heart.

FIG. 2 shows a distal portion of a lead according to one embodiment.

FIG. 3 shows a distal portion of a lead according to one embodiment.

FIG. 4 shows a distal portion of a lead according to one embodiment.

FIG. 5 shows a view of a lead, according to one embodiment, implanted within a heart.

FIG. 6 shows a front view of a lead according to one embodiment.

FIG. 7 shows an intermediate portion of a lead according to one embodiment.

FIG. 8 shows a view of a lead, according to one embodiment, implanted within a heart.

FIG. 9 shows a view of a lead, according to one embodiment, implanted within a heart.

FIG. 10 shows a view of a lead, according to one embodiment.

FIG. 11 shows a view of a lead, according to one embodiment.

FIG. 12 shows a view of a lead, according to one embodiment.

FIG. 13 shows a view of a lead, according to one embodiment, implanted within a heart.

FIG. 14 shows a view of a lead, according to one embodiment, implanted within a heart.

FIG. 15 shows a view of a lead, according to one embodiment, implanted within a heart.

FIG. 16 shows a view of a lead, according to one embodiment, implanted within a heart.

FIG. 17 shows a cross-section of a lead in accordance with one embodiment.

FIG. 18 shows a cross-section of a lead in accordance with one embodiment.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

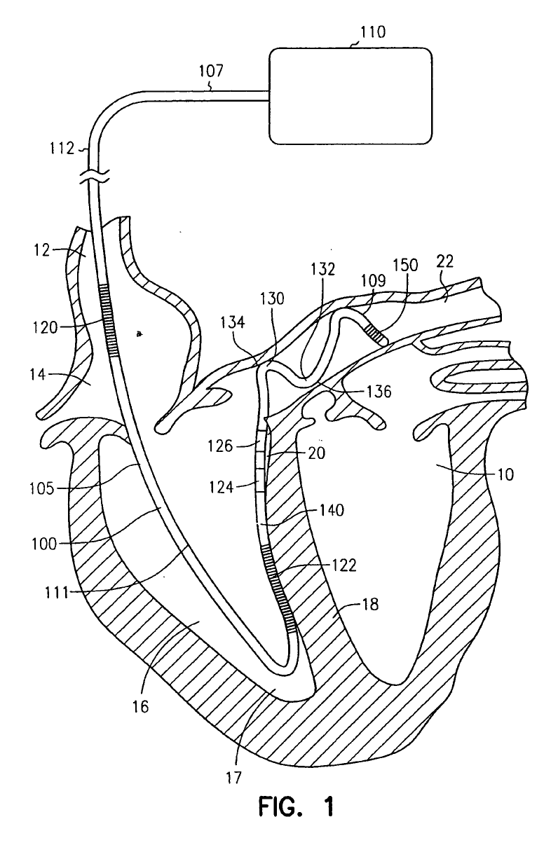

FIG. 1 shows a view of a lead 100 implanted within a heart 10. Heart 10 generally includes a superior vena cava 12, a right atrium 14, a right ventricle 16, a right ventricular apex 17, a ventricular septum 18, and a ventricular outflow tract 20, which leads to a pulmonary artery 22. In one embodiment, lead 100 is adapted to deliver defibrillation shocks to heart 10. Lead 100 is part of an implantable system including a pulse generator 110, such as a defibrillator.

Pulse generator 110 can be implanted in a surgically-formed pocket in a patient's chest or other desired location. Pulse generator 110 generally includes electronic components to perform signal analysis, processing, and control. Pulse generator 110 can include a power supply such as a battery, a capacitor, and other components housed in a case. The device can include microprocessors to provide processing and evaluation to determine and deliver electrical shocks and pulses of different energy levels and timing for ventricular defibrillation, cardioversion, and pacing to heart 10 in response to cardiac arrhythmia including fibrillation, tachycardia, and bradycardia.

In one embodiment, lead 100 includes a lead body 105 extending from a proximal end 107 to a distal end 109 and having an intermediate portion 111. Lead 100 includes one or more conductors, such as coiled conductors or other conductors, to conduct energy from pulse generator 110 to heart 10, and also to receive signals from the heart. The lead further includes outer insulation 112 to insulate the conductor. The conductors are coupled to one or more electrodes, such as electrodes 120, 122, 124, and 126. Lead terminal pins are attached to pulse generator 110. The system can include a unipolar system with the case acting as an electrode or a bipolar system with a pulse between two of the electrodes.

In one embodiment, lead 100 is adapted for septal placement of one or more of the electrodes while utilizing pulmonary artery 22 for lead fixation. By using the pulmonary artery, the lead can be implanted such that the electrode contacts the upper portion of septum 18 above apex 17 without requiring active fixation. Lead 100 can thus shock, pace, and sense at the interventricular septum 18 or ventricular outflow tract 20.

For example, in one embodiment electrode 122 is disposed along intermediate portion 111 of the lead. Electrode 122 can be a defibrillation electrode, such as a coil defibrillation electrode designed to deliver a defibrillation shock of approximately 3 joules to approximately 60 joules to septum 18 from the pulse generator. Electrode 122 can also deliver cardioversion shocks of approximately 0.1 joules to approximately 10 joules. In one example, electrode 122 can be a spring or coil defibrillation electrode.

When present leads are inserted in the heart and positioned such that an electrode is against the high ventricular septum (above the apex 17), the leads require active fixation. However, active fixation can cause repeated trauma to the endocardial tissue because of the cyclical motion of the heart, and thus may have possible micro-dislodgement and increase defibrillation and pacing thresholds.

In one embodiment of the present system, distal end 109 of lead 100 includes a pre-formed, biased shape 130 adapted to passively fixate distal end 109 of the lead within pulmonary artery 22 with electrode 122 positioned in the right ventricle at a high septal location or ventricular outflow tract. In one embodiment, pre-formed, biased shape 130 includes an S-shaped configuration 132. The pre-formed, biased shape 130 generally includes at least two lead surfaces (such as surfaces 132 and 136, for example) which are dimensioned and positionable such that the surfaces contact opposing walls of the pulmonary artery.

In various embodiments, pre-formed bias shape 130 can include a curved shape such as an S-shape, a C-shape, a J-shape, an O-shape, and other non-linear shapes adapted for contacting one or more sides of the pulmonary artery to provide sufficient fixation of the lead. Such a design is more reliable because the lead becomes easier to implant and explant because of the passive fixation which is allowed by the shape of distal portion of lead 100. Moreover, passive fixation allows for easier adjustment of the electrode placement. Also, there is less trauma or perforation to endocardium tissue, which can yield lower pacing thresholds, and there is less trauma to the high septal or outflow tract than caused by active fixation at the high septal or outflow tract location. To form pre-formed biased shape 130, the lead body can be manufactured in the pre-biased shape or the conductor coil can be formed in the pre-biased shape to thus bias the lead body.

In one embodiment, electrodes 124 and 126 of lead 100 can include pacing/sensing electrodes, such as ring electrodes located distally from electrode 122. Electrodes 124 and 126 are proximal from distal end 109 and are located on the lead to sense or pace at the ventricular septum or the ventricular outflow tract when the lead is implanted.

In one embodiment, electrode 120 includes a second coil defibrillation electrode acting as a return electrode for electrode 122 in a bipolar system. Electrode 120 can be positioned in superior vena cava 12 or right atrium 14.

In one embodiment, at least a portion of lead 100 can include an anti-thrombosis coating 140, such as Hypren or polyethleneglycol for example. Coating 140 can be placed on the lead, for example on one or more of the distal electrodes 122, 124, 126, or on other segments of the lead.

In one embodiment, lead 100 can include a sensor 150, such as a cardiac output sensor, mounted proximate a distal segment of the lead or mounted on the intermediate portion of the lead. Sensor 150 is implanted to a location within the pulmonary artery or within the outflow tract 20 to monitor cardiac output through pulmonary artery 22. For example, a cardiac output monitoring sensor 150 can be placed proximate the distal end of the lead to measure cardiac output through the pulmonary artery. Sensor 150 can be coupled to pulse generator 110 through a conductor.

In one embodiment, sensor 150 can be a flow speed sensor, allowing the system to know how fast the blood is going through the artery. For example, sensor 150 can be a metal ring or coil. Such a component would have resistance properties such that if a pulse of energy was sent through the component, the component would heat up, which would in turn increase the electrical resistance of the component. The electrical resistance could be monitored over time to determine how it changes as the blood flow going past it cools it down to blood temperature. The faster the blood flow, the faster the component will cool down and hence the faster the resistance should drop. This cool down or resistance change can be correlated to the blood flow. In other embodiments, sensor 150 can be a pressure sensor. In some embodiments, sensor 150 can include a CO₂ or O₂ sensor.

In these embodiments, sensor 150 can be used to determine blood flow to allow the position of electrodes 122, 124, and 126 to be optimized. For example, the cardiac output can be used to change the position of the electrode either during or after implantation. In some examples, sensor 150 can be used to help optimize the location of other electrodes on separate leads located within the heart. Moreover, sensor 150 can be used to provide pacing and sensing information to the pulse generator to deliver pulses or modify the settings of the pulse generator.

In some embodiments, lead 100 can be configured to allow both a stylet or catheter delivery. For example, an opening can be left through the middle of the lead to allow a stylet to be used.

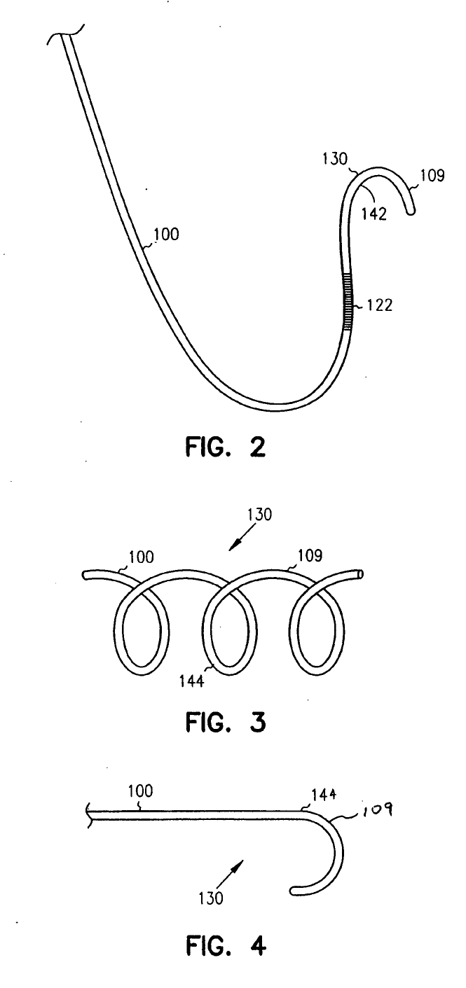

FIG. 2 shows distal portion 109 of lead 100 according to one embodiment. In this example, pre-formed, biased shape 130 includes a J-shaped curve 142 at a distal tip of the lead body. J-shaped curve 142 can be positioned within pulmonary artery 22 (FIG. 1) or in one of the branch arteries off of the pulmonary artery to passively fixate the distal end of the lead within the pulmonary artery.

FIG. 3 shows distal portion 109 of lead 100 according to one embodiment. In this example, pre-formed, biased shape 130 includes a spiral configuration 144.

FIG. 4 shows distal portion 109 of lead 100 according to one embodiment. In this example, pre-formed, biased shape 130 includes a C-shaped configuration 144.

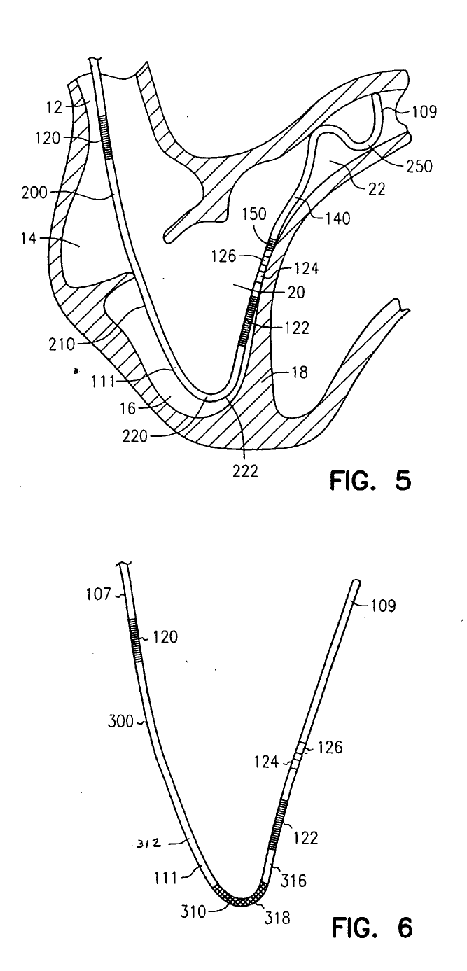

FIG. 5 shows a view of a lead 200 according to one embodiment. Lead 200 includes some of the components discussed above for lead 100, and the above discussion is incorporated herein. Lead 200 is implanted in heart 10 (FIG. I) with distal end 109 located within pulmonary artery 22 and electrode 122 positioned against septum 18 or within ventricular outflow tract 20.

In one embodiment, lead 200 includes a lead body 210 including a pre-formed V-shape or J-shape 220 formed in the intermediate portion 111 of the lead body. J-shape 220 is located such that electrode 122 is located distally from a bottom 222 of the pre-formed J-shape 220. Various embodiments includes a pre-formed J-shape in either 2D or 3D. J-shaped portion 220 of lead 200 allows for better septal/electrode contact. To pre-form the lead, the lead can be manufactured such that it is biased in the J-shape. Thus, the lead naturally reverts to the J-shape when it is implanted. For example, the lead body can be formed in the pre-biased shape or the conductor coils can be formed in the pre-biased shape to bias the lead body into the shape. When implanted, the bottom 222 of the J-shape 220 is within the right ventricle 16 and electrode 122 is positioned proximate ventricular septum 18 or right ventricular outflow tract 20 such that at least a portion of the distal end 109 of the lead body is located within a pulmonary artery 22. The pre-formed J-lead design enhances the septal electrode stability and contact, and can help result in lower defibrillation and pacing thresholds because of better electrode contacts.

In one embodiment, a second electrode 120 is located proximally from the bottom 222 of the J-shape and positioned to be located within superior vena cava 12 or right atrium 14 when the distal end 109 of the lead is within the pulmonary artery 22. Lead 200 can also include one or more pacing/sensing electrodes 124, 126 located distally from electrode 122 to sense or pace at the ventricular septum 18 or the ventricular outflow tract 20. One embodiment includes a sensor 150, such as a cardiac output sensor. In this example, sensor 150 is located within the outflow tract 20.

In one embodiment, distal end 109 is adapted for being fixated within a pulmonary artery. One embodiment provides a passive fixation technique, as described above in FIGS. 1-4. For example, a pre-formed biased distal portion 250 can be provided. In some embodiments, to be discussed below, an active fixation technique is utilized. Some embodiments utilize neither passive nor active fixation, relying on the J-shape 220 and gravity to hold the electrodes 122, 124, and 126 in place against the septum or the outflow tract.

FIG. 6 shows a front view of a lead 300 according to one embodiment. Lead 300 includes some of the components discussed above for leads 100 and 200, and the above discussion is incorporated herein. Lead 300 can be implanted in a heart with distal end 109 located within the pulmonary artery and electrode 122 positioned against the septum or within the ventricular outflow tract.

In one embodiment, lead 300 includes a section 310 of the intermediate section 111 of the lead which is less stiff, or more pliable, than adjacent sections 312 and 316 of the lead body. Less stiff section 310 is located proximally from electrode 122 and distally from electrode 120. When lead 300 is positioned in the heart with distal portion 109 in the pulmonary artery, the soft, or less stiff section 310 allows the lead to naturally fall into place and contact the septum due to gravity. Lead 300 is adapted to be placed within a heart in a J-shaped configuration with the less stiff section 310 near a bottom 318 of the J-shape such that electrode 122 is positioned proximate a ventricular septum or a right ventricular outflow tract and at least a portion of the distal end 109 of the lead body is located within a pulmonary artery. The less stiff section 310 helps reduce any forces caused by heart motion to be transferred to a site of the septal electrode.

In one embodiment, the less stiff section 310 includes a different, more pliable material than the material of adjacent sections 312 and 316. Again, when the lead is positioned in the heart, the softer segment allows the lead to naturally fall into place and contact the septum due to gravity, and thus enhances the septal electrode stability and contact and reduces or eliminates the forces and motion (caused by heart motion) transferred to the site of the septal electrode 122. This can result in lower defibrillation and pacing thresholds because of better electrode contact.

In this example, no fixation technique is shown in the pulmonary artery for lead 300. In other embodiments, a passive technique as shown above in FIGS. 1-5, or the active technique discussed below can be utilized in conjunction with this embodiment.

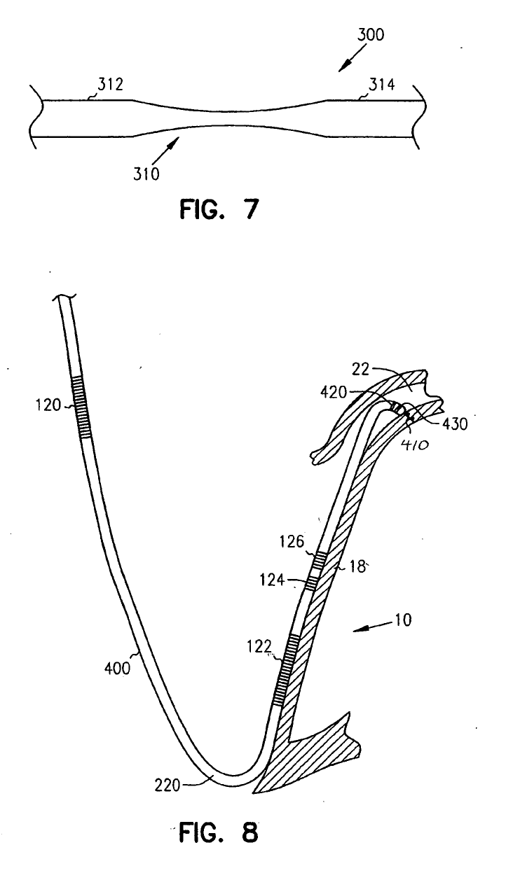

FIG. 7 shows a portion of lead 300 according to one embodiment. In this embodiment, less stiff section 310 includes a smaller diameter than the adjacent sections 312 and 314. The smaller diameter section 310 is more flexible than the adjacent thicker regions.

In other embodiments, less stiff section 310 can be formed by providing a lead wall having a different internal diameter thickness, or by providing a less stiff conductor coil at that location.

Referring to FIG. 1, in one example use of one or more of the leads discussed herein, the lead is inserted through the right ventricle 16 and into the pulmonary artery 22 using a guiding catheter or a stylet. The lead is positioned until the distal end of the lead is in the pulmonary artery and electrodes 122, 124, and 126 are positioned against the septum or within the outflow tract. The distal end of the lead can be fixated within the artery by one of the techniques discussed above. The pulse generator can be used to sense the activity of the heart using electrodes 124 and 126, for example. When there is need for a cardioversion or defibrillation shock, the shock is delivered via electrode 122. As discussed, in various examples, the lead body can be configured in a pre-formed J-shape such that shock electrode is located distally from a bottom of the J-shape, or a less stiff section can be provided.

FIG. 8 shows a view of a lead 400 according to one embodiment, implanted within a heart 10. Lead 400 is adapted to be actively fixated within the pulmonary artery 22 utilizing a helix 410, or other fixation mechanism. In one embodiment, lead 400 includes radiopaque markers 420 near the distal tip to help a physician guide the lead when viewed under fluoroscopy. One embodiment includes a drug elution member 430, which can elude steroids, for example, to reduce inflammatory response of the tissue. In some embodiments, lead 400 does not include either the pre-formed J-shape 220 (FIG. 5) or the less stiff section 310 (FIG. 6) of the leads discussed above. Lead 400 can be an unbiased, flexible lead relying on helix 410 for fixation within the pulmonary artery. In other embodiments, the active fixation technique can be used with the leads discussed above. In some embodiments, active fixation can be provided in addition to or in place of the passive fixation design discussed above.

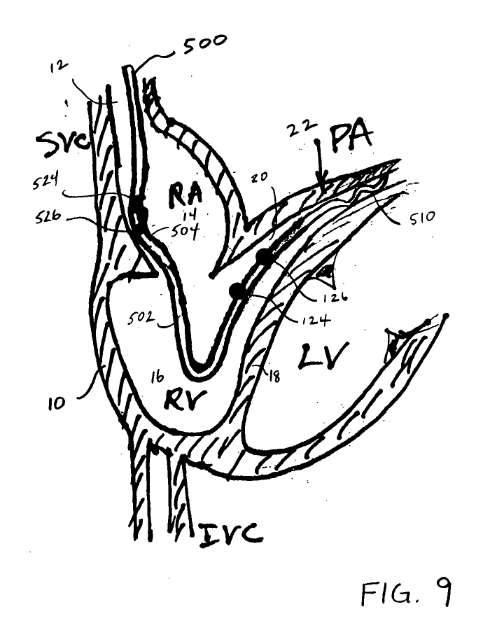

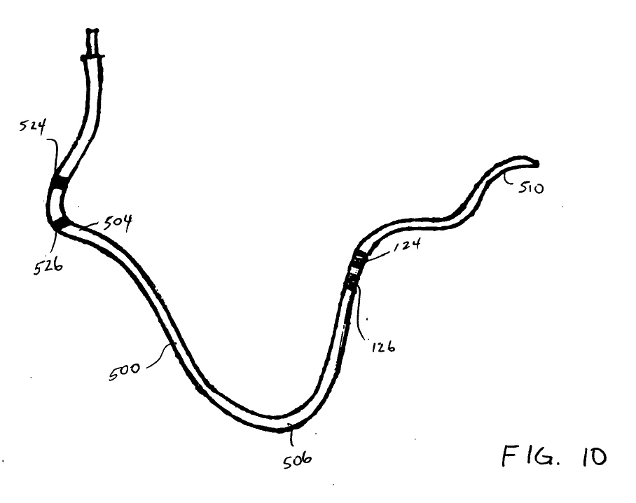

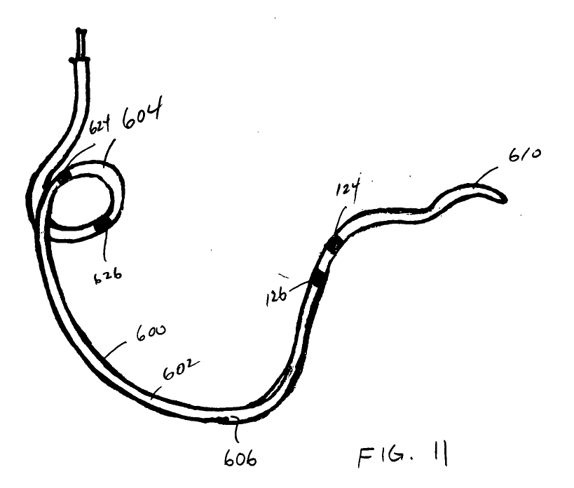

FIG. 9 shows a view of a lead 500 according to one embodiment, implanted within a heart 10. Lead 500 is a single-pass lead adapted to be passively or actively fixated within pulmonary artery 22 utilizing a fixation mechanism such as a biased shape distal end 510, or other passive or active technique as discussed above. Lead 500 includes a lead body 502 and electrodes 124, 126 which are located so as to be proximate to or abut the septum 18 or be within the outflow tract 20.

The lead body 502 also has one or more electrodes 524, 526. Electrodes 524, 526 are adapted for positioning and/or fixation to the wall of atrium 14 of the heart. A passive fixation element can be used as part of the second electrode or electrode pair. For example, in one embodiment lead body 502 also includes a curved portion 504 which facilitates the positioning and fixing of electrodes 524, 526 to the right atrium. Curve 504 is positioned in the right atrium 14 of the heart after implantation, and positions the electrode(s) 524, 526 closer to the wall of the atrium to enhance the sensing and pacing performance of the lead.

In one embodiment, electrodes 524, 526 are adapted for delivering atrial pacing therapy. Electrodes 524, 526 can also be used for atrial sensing. Curved portion 504 of lead 500 positions the atrial electrodes 524, 526 closer to the wall of the heart in the right atrium 14. This enhances electrical performance as the electrodes will be closer to the portion of the heart where the signal will pass.

The shape of the biased or curved portion 504 facilitates the placement of the atrial electrode against the atrial wall during implantation. The shape of the lead will also be approximately the same before implantation as after implantation and the result will be that the shape reduces the nominal residual stresses in the lead body 500.

Electrodes 524, 526 can be ring electrodes which can be exposed, or partially masked by the lead body. In some embodiments, the electrodes can be hemispherical tip electrodes. In some embodiments, the electrodes can have a porous surface to help fixation to the atrium.

Lead 500 can be implanted as discussed above such that electrodes 124, 126 are located in the outflow tract 20 or adjacent the RV septum 18. Electrodes 524, 526 are then located in the right atrium. Such an embodiment allows for RV septal pacing as discussed above. It further allows for right atrium pacing and/or sensing using the single-pass lead 500.

The single-pass lead 500 equipped with atrial electrodes 524, 526 is capable of being fixed to the endocardial wall allowing for better sensing capability and better current delivery to the heart. Electrodes 524, 526 can be placed on the outside of the curved portion of the lead body. The fixed atrial electrode(s) enhance lead stabilization within the heart. This results in no need for two leads in the heart, while allowing for a pacing system to detect and correct an abnormal heartbeat in both the atrium and ventricle, which may have independent rhythms.

In some embodiments, the lead can include steroid elution from any of the electrodes 124, 126, 524, and 526. Drug elution, typically steroid, can be provided by using one or more of the drug-releasing technologies such as sleeves or collars positioned in close proximity to the electrodes or by the use of internalized drug-containing plugs. An example of the composition of at least one collar is dexamethasone acetate in a simple silicone medical adhesive rubber binder or a steroid-releasing plug similarly fabricated.

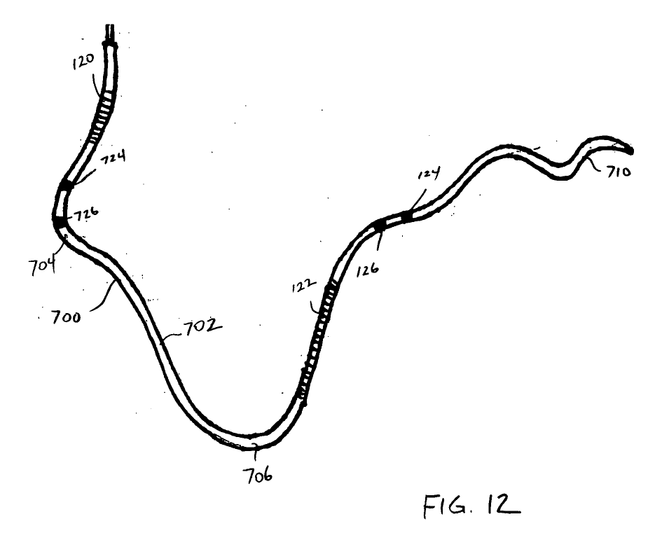

FIG. 10 shows further details of lead 500, in accordance with one embodiment. Lead 500 can include a preformed or biased curved portion 506 on a mid-portion of the lead. Curved portion 506 can be a pre-formed portion of the lead or a more flexible area of the lead, such as discussed above.

FIG. 11 shows a lead 600, in accordance with one embodiment. Certain details of lead 600 are similar to lead 500 and the above discussion is incorporated by reference. Lead 600 is a single-pass lead adapted to be passively or actively fixated within the pulmonary artery utilizing a fixation mechanism such as a biased shape distal end 610, or other passive or active technique as discussed above. Lead 600 includes a lead body 602 and electrodes 124, 126 which are located so as to be proximate to or abut the septum or be within the outflow tract when implanted.

The lead body 602 also has one or more electrodes 624, 626. Electrodes 624, 626 are adapted for positioning and fixation to the wall of the atrium of the heart. In one embodiment, lead body 602 includes also includes a curved portion 604 which facilitates the positioning and fixing of electrodes 624, 626 to the right atrium. Curved portion 604 is positioned in the right atrium of the heart after implantation, and positions the electrode(s) 624, 626 closer to the wall of the atrium to enhance the sensing and pacing performance of the lead. In this example, curved portion 604 includes a looped or spiral curve.

In one embodiment, electrodes 624, 626 are adapted for delivering atrial pacing therapy. Electrodes 624, 626 can also be used for atrial sensing. Curved portion 604 of the lead 600 positions the atrial electrodes 624, 626 on the curved portion or biased section 604 closer to the wall of the heart in the right atrium. This enhances electrical performance as the electrodes will be closer to the portion of the heart where the signal will pass.

FIG. 12 shows a lead 700 in accordance with one embodiment. Lead 700 includes a lead body 702 and one or more conductors, such as coiled conductors or other conductors, to conduct energy from a pulse generator to a heart: The conductors are coupled to one or more electrodes, such as electrodes 120, 122, 124, 126, 724, and 726. The system can include a unipolar system with the pulse generator case acting as an electrode or a bipolar system with a pulse between two of the electrodes.

In one embodiment, lead 700 is adapted for septal placement of one or more of the electrodes while utilizing the pulmonary artery for lead fixation. Lead 700 can thus shock, pace, and sense at the interventricular septum or ventricular outflow tract or in the right atrium or superior vena cava.

For example, in one embodiment electrode 122 is disposed along an intermediate portion of the lead. As discussed above, electrode 122 can be a defibrillation electrode, such as a coil defibrillation electrode designed to deliver a defibrillation shock of approximately 3 joules to approximately 60 joules to septum 18 from the pulse generator. Electrode 122 can also deliver cardioversion shocks of approximately 0.1 joules to approximately 10 joules. In one embodiment, electrode 120 includes a second coil defibrillation electrode acting as a return electrode for electrode 122 in a bipolar system. Electrode 120 can be positioned in the superior vena cava or right atrium.

Preformed or biased curved portions 704 and 706 can be structured as discussed above. Portions 704 and 706 can be 2-dimensional curves or 3-dimensional curves.

Lead 700 can be used for one or more of the following therapies: RV septal pacing and RA pacing; RV septal pacing, RV pacing and RV shocking; RV septal pacing, RA pacing, RV shocking, and RA/superior vena cava shocking.

The single pass lead 700 permits the ability to utilize a single lead for a variety of bradyarrythmia and tachyarrythmia therapies and also for treating CHF.

In one embodiment, lead 700 includes four independent conductors coupled to respective electrodes. For example, electrodes 122 and 126 can be electrically connected to a single conductor and electrodes 120 and 724 can be coupled to a single conductor, with electrodes 726 and 124 coupled to respective conductors. This allows for a smaller diameter lead and better reliability than if each electrode had its own conductor.

In one example,. lead 700 can be implanted by a stylet, over-the-wire, or catheter technique, including first inserting a distal biased portion 710 of the lead into the pulmonary artery. Then the location of the septal pacing electrodes 124, 126 can be adjusted by further maneuvering of the distal portion 710. Once the electrodes 124, and 126 are properly positioned, the distal portion is fixed in the pulmonary artery, as discussed above. Coil electrode 122 is positioned so it is against the septum, the right atria electrodes 724, 726 are positioned in the right atrium.

In one or more examples discussed above, the single pass system allows the lead to detect and correct an abnormal heartbeat in both the atrium and ventricle which may have independent rhythms, as well as a defibrillation system to detect and correct an abnormally fast heart rate (tachycardia condition). The system also allows for synchronized pacing.

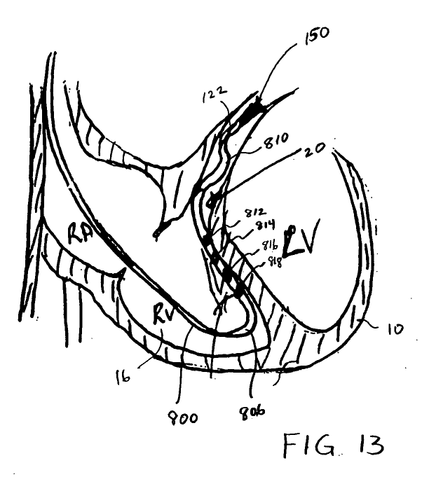

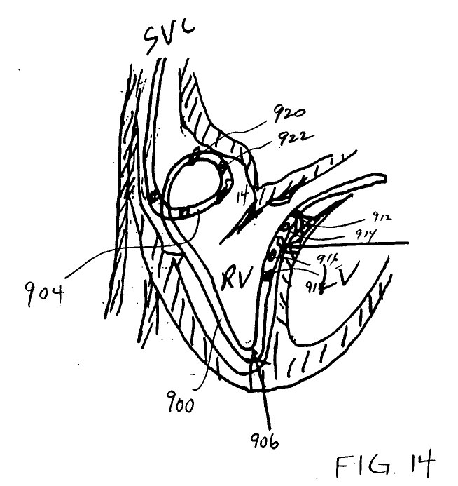

FIG. 13 shows a lead 800, according to one embodiment. In one embodiment, lead 800 is adapted for CHF therapy and for the prevention of sudden cardiac death (SCD). Lead 800 includes a cardiac output sensor 150, such as discussed above. Lead 800 also includes a biased portion 810 on a distal end for fixation within pulmonary artery. 122, in a manner as discussed above. In this example, lead 800 includes electrodes 812, 814, 816, and 818 on an intermediate portion of the lead body. Electrodes 812-818 can be ring electrodes, for example. Electrodes 812-818 are located on the lead so that the electrodes are positioned proximate or adjacent the ventricular septum or the RV outflow tract 20, when the lead is implanted. A section 806 of lead 800 can provide a pre-formed J-shape, as discussed above.

Electrodes 812-818 are used to deliver energy to the heart at a specific location. In use, a physician tests each electrode independently to ascertain which electrode or electrodes are correctly located to deliver energy to the “sweet-spot” of the heart. The “sweet-spot” is the location on the septum/outflow tract which is optimal for pacing. In some embodiments, lead 800 can include two, four, six, eight, or more electrodes having various spacing between the electrodes along the length of the lead.

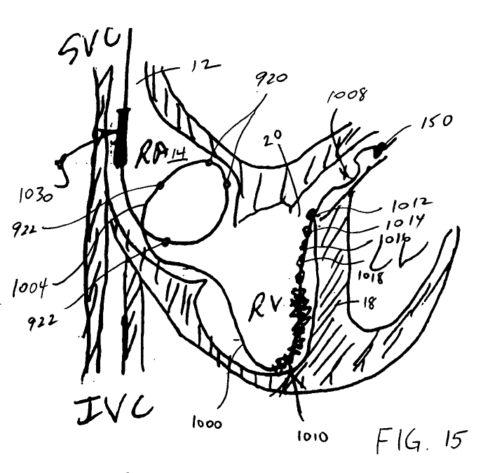

FIG. 14 shows a lead 900 in accordance with one embodiment. Lead 900 includes a plurality of electrodes 912, 914, 916, and 918, for septum/outflow tract pacing as discussed above. Lead 900 also includes two or more proximal electrodes 920, 922, for example, which are positioned on the lead so as to be located in the right atrium 14 when the lead is implanted. Lead 900 can include a preformed, biased shape 904, such as a loop or C-shape to help bias electrodes 920, 922 towards the atrium walls. A section 906 of lead 900 can be pre-formed or less stiff to provide a J-shape, as discussed above.

FIG. 15 shows a lead 1000, in accordance with one embodiment. Lead 1000 includes a plurality of electrodes 1012, 1014, 1016, and 1018, for septum/outflow tract pacing/sensing, as discussed above. The lead can include electrodes 920, 922 located on a pre-formed, biased section 1004 to be locatable within the right atrium, as discussed above. The lead can also include a preformed distal end 1008 for pulmonary artery fixation, and a cardiac output sensor 150. In this embodiment, lead 1000 includes a shocking electrode, such as coil electrode 1010 located on the lead so as to be proximate the ventricular septum 18 or the ventricular outflow tract 20. Lead 1000 also includes a shocking electrode, such as a coil electrode 1030 located so as to be within superior vena cava 12 or right atrium 14.

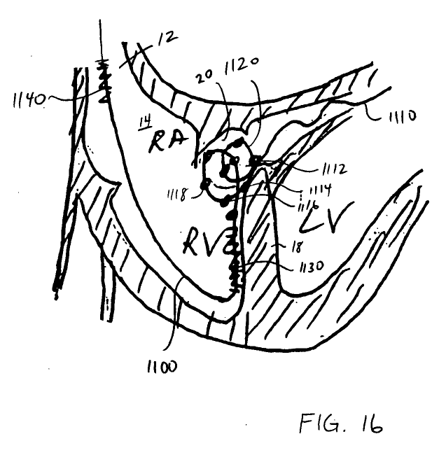

FIG. 16 shows a lead 1100, in accordance with one embodiment. Lead 1100 includes a distal biased portion 1110 to help fixate the lead in the pulmonary artery. In this embodiment, lead 1100 includes a shocking electrode, such as coil electrode 1130 located on the lead so as to be proximate the ventricular septum 18 or the ventricular outflow tract 20. Lead 1100 also includes a second shocking electrode, such as a coil electrode 1140 located so as to be within superior vena cava 12 or right atrium 14.

In one embodiment, lead 1100 includes a pre-formed biased intermediate portion 1120. Biased portion 1120 can be a pre-formed spiral shape, for example. The biased potion is located so as to be within the outflow tract 20 when the lead is implanted. Two or more electrodes 1112, 1114, 1116, 1118, are disposed along the lead at biased portion 1120. The biased portion biases the electrodes towards the heart tissue of the outflow tract to ensure better electrode/tissue contact. The configuration allows lead 1100 to deliver energy to the “sweet-spot” of the heart. Again, the “sweet-spot” is the location on the septum/outflow tract which is optimal for pacing.

In any of the embodiments of FIGS. 13-16, the lead can include 2, 4, 8, or more electrodes to help locate the optimal septal/outflow tract pacing site, or “sweet-spot.” The multiple electrodes also can also be used for mapping the activity of the heart. Also, some embodiments can use either passive or active fixation within the pulmonary artery. The examples can include electrodes for right atrium pacing/sensing, as well as shocking electrodes for RV septal shocking and/or RA/SVC shocking.

In further embodiments, the leads discussed above can include an anti-thrombosis coating on the lead or electrodes, the leads can be iso-diameter or non-isodiameter, and implantation can be by stylet or catheter, as discussed above.

The leads of FIGS. 13-16 are especially applicable to CHF therapy. The leads, with fixation in the pulmonary artery are easier to implant than leads going into the coronary sinus. Moreover, utilizing the RV septal/outflow tract area is effective for treating CHF patients, especially if the “sweet spot” is located. In some embodiments, the present leads are adapted to be fixated in the pulmonary artery and used to locate the sweet spot by using a plurality of electrodes, which are independently operable so they can be individually checked by the physician to determine the optimal pacing location.

Any of the leads can include a cardiac output sensor. As discussed above, the cardiac output sensor can be used to determine blood flow to allow the position of the distal electrodes to be optimized. For example, the cardiac output can be used to change the position of the electrode either during or after implantation. In some examples, the cardiac output sensor can be used to help optimize the location of other electrodes on separate leads located within the heart. Moreover, the cardiac output sensor can be used to provide pacing and sensing information to the pulse generator to deliver pulses or modify the settings of the pulse generator.

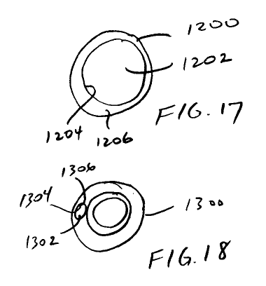

FIG. 17 shows a schematic representation of a cross-section of a lead 1200 according to one embodiment. In various embodiments, lead 1200 can include any of the lead configurations discussed above. Lead 1200 includes a lumen 1202 extending through the entire length of the lead. In one embodiment, lumen 1202 is defined by the inner surface 1204 of a conductor coil 1206. Lumen 1202 facilitates inserting any of the leads discussed above using an over-the-wire technique. To insert an over-the-wire lead, a guide wire is inserted to the desired location, such as into the pulmonary artery. The lead is then fed over the wire such that the wire is within the lumen of the lead, until the lead reaches the proper location. The guide wire is removed. If the lead has any biased, pre-formed shaped section, such as described above, those sections return to their biased configuration. For example, some embodiments above included leads having distal ends having a biased configuration. Such as lead would expand to its original shape to fixate the distal end of the lead in the pulmonary artery.

FIG. 18 shows a schematic representation of a cross-section of a lead 1300 according to one embodiment. In various embodiments, lead 1300 can include any of the lead configurations discussed above. Lead 1300 includes a lumen 1302 extending through the entire length of the lead. In one embodiment, lumen 1302 is defined by the inner surface 1304 of a formed polymer passage 1306. Lumen 1302 facilitates inserting any of the leads discussed above, in an over-the-wire configuration such as discussed above. In some embodiments, lumen 1302 can be centered or off-center.

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. A lead comprising:
a lead body extending from a proximal end to a distal end and having an intermediate portion; and
a first electrode disposed along the intermediate portion of the lead body;
wherein the distal end of the lead body includes a pre-formed, biased shape adapted to passively fixate the distal end of the lead body within a pulmonary artery with the first electrode positioned against the ventricular septum or ventricular outflow tract;
the lead body having a curved portion and a second electrode disposed along the curved portion, wherein the second electrode is positioned a distance from the first electrode such that the second electrode is within a right atrium when the first electrode is positioned against the ventricular septum or ventricular outflow tract.

2. The lead of claim 1, wherein the pre-formed, biased shape includes an S-shaped configuration.

3. The lead of claim 1, wherein the pre-formed, biased shape includes a C-shaped configuration.

4. The lead of claim 1, wherein the pre-formed, biased shape includes a spiral configuration.

5. The lead of claim 1, wherein the pre-formed, biased shape includes a J-shaped curve at a distal tip of the lead body.

6. The lead of claim 1, wherein the preformed, biased shape includes at least two surfaces positioned to contact opposing walls of the pulmonary artery.

7. The lead of claim 1, wherein the pre-formed, biased shape includes at least one curve in the lead body dimensioned such that at least two lead surfaces on the distal end of the lead contact at least two walls of the pulmonary artery.

8. The lead of claim 1, wherein the lead body further includes a preformed J-shape, wherein the electrode is located distally from a bottom of the pre-formed J-shape.

9. The lead of claim 1, wherein a section of the intermediate portion of the lead body is less stiff than adjacent sections of the lead body, the less stiff section located proximally from the first electrode.

10. The lead of claim 1, wherein first electrode includes a defibrillation coil electrode.

11. The lead of claim 1, further comprising a third electrode located distally from the first electrode.

12. The lead of claim 1, further comprising a sensor mounted to the distal end.

13. The lead of claim 1, wherein the lead body includes a lumen through an entire length of the lead body, such that the lead can be implanted over a guide wire.

14. A lead comprising:
a lead body extending from a proximal end to a distal end and having an intermediate portion; and
at least two electrodes disposed along the intermediate portion of the lead body;
wherein the distal end of the lead body is adapted to be passively fixated within a pulmonary artery and the at least two electrodes are positioned on the lead such that the at least two electrodes are located proximate a ventricular septum or ventricular outflow tract when the distal end is in the pulmonary artery.

15. The lead of claim 14, wherein the distal end of the lead body includes a pre-formed, biased shape.

16. The lead of claim 14, wherein the lead body includes at least four electrodes.

17. The lead of claim 14, wherein the lead body includes a proximal electrode positioned a distance from the at least two electrodes such that the proximal electrode is within the right atrium when the at least two electrodes are positioned against the ventricular septum or ventricular outflow tract.

18. The lead of claim 17, wherein the lead body includes a curved portion and the proximal electrode is located on the curved portion.

19. The lead of claim 14, wherein the two or more electrodes are independently operable to allow for one or more optimally positioned electrodes to be used for delivering energy to the heart.

20. The lead of claim 14, further comprising a sensor mounted to the distal end of the lead body.

21. The lead of claim 14, further including a defibrillation coil electrode located on an intermediate portion of the lead body.

22. The lead of claim 21, wherein the coil electrode is located on the lead body such that the coil electrode is proximate the ventricular septum or the ventricular outflow tract when the distal end of the lead is within the pulmonary artery.

23. The lead of claim 14, further including a defibrillation coil electrode located on an intermediate portion of the lead body such that the coil electrode is proximate the superior vena cava or the right atrium when the distal end of the lead is within the pulmonary artery.

24. The lead of claim 14, wherein the two or more electrodes are located on a pre-formed biased portion of the lead body.

25. The lead of claim 24, wherein the pre-formed biased portion includes a spiral shape.

26. The lead of claim 14, wherein the lead body includes a lumen through an entire length of the lead body, such that the lead can be implanted over a guide wire.

27. The lead of claim 26, wherein the lumen is defined by a conductor coil of the lead.

28. The lead of claim 26, wherein the lumen is defined by a passage through a material of the lead body.

29. A method comprising:
providing a lead having a lead body extending from a proximal end to a distal end and having an intermediate portion, the lead body having a first electrode disposed along the intermediate portion, wherein the distal end of the lead body includes a preformed, biased shape adapted to passively fixate the distal end of the lead body within a pulmonary artery, the lead body having a curved portion and a second electrode disposed along the curved portion; and
inserting the lead body through a right ventricle and into a pulmonary artery; and
disposing the first electrode proximate to a ventricular septum or a ventricular outflow tract and passively fixating the distal end within the pulmonary artery and disposing the second electrode within a right atrium.

30. The method of claim 29, further comprising delivering pacing energy pulses from the first electrode.

31. The method of claim 29, further comprising delivering pacing energy pulses from the first electrode.

32. The method of claim 29, further comprising providing a shocking electrode on the intermediate portion of the lead body and located so to be proximate the ventricular septum or the ventricular outflow tract when the distal end is within the pulmonary artery.

33. The method of claim 29, further comprising providing a shocking electrode on the intermediate portion of the lead body and located so to within the right atrium or a superior vena cava when the distal end is within the pulmonary artery.

34. The method of claim 29, wherein inserting the lead body includes inserting the lead body such that the preformed, biased shape includes at least two surfaces positioned to contact opposing walls of the pulmonary artery when the lead is implanted.

35. The method of claim 29, wherein inserting the lead body includes inserting the lead body over a guide wire.

36. A method comprising:
providing a lead having a lead body extending from a proximal end to a distal end and having an intermediate portion, the lead having at least two electrodes disposed along the intermediate portion, wherein the distal end of the lead is adapted to be passively fixated within a pulmonary artery; and
inserting the lead body through a right ventricle and into a pulmonary artery and disposing the at least two electrodes proximate a ventricular septum or ventricular outflow tract.

37. The method of claim 36, including passively fixating the distal end within the pulmonary artery with a pre-formed, biased shape.

38. The method of claim 36, including independently operating the two or more electrodes to determine an optimal location proximate the ventricular septum or ventricular outflow tract for pacing.

39. The method of claim 36, including providing at least four electrodes disposed along the intermediate portion.

40. The method of claim 36, including providing at least eight electrodes disposed along the intermediate portion.

41. The method of claim 36, including providing the lead body with a proximal electrode positioned a distance from the at least two electrodes such that the proximal electrode is within the right atrium when the at least two electrodes are positioned against the ventricular septum or ventricular outflow tract.

42. The method of claim 41, including providing the lead body with a curved portion and the proximal electrode is located along the curved portion.

43. The method of claim 36, including providing a sensor mounted to the distal end of the lead body to monitor cardiac output through the pulmonary artery.

44. The method of claim 36, including providing a defibrillation coil electrode on an intermediate portion of the lead body such that the coil electrode is proximate the ventricular septum or the ventricular outflow tract when the distal end of the lead body is within the pulmonary artery.

45. The method of claim 36, including providing a defibrillation coil electrode located on an intermediate portion of the lead body such that the coil electrode is proximate the superior vena cava or the right atrium when the distal end of the lead body is within the pulmonary artery.

46. The method of claim 36, including providing pre-formed biased portion of the lead, wherein the two or more electrodes are located along the pre-formed biased portion.

47. The method of claim 36, further comprising delivering pacing energy pulses from at least one of the at least two electrodes.

48. The method of claim 36, wherein inserting the lead body includes inserting the lead body over a guide wire.
